(19) United States
(12) Patent Application Publication
(10) Pub. No.: US 2020/0215260 A1
(43) Pub. Date: Jul. 9, 2020

(54) MEDICAL SPACER FOR MANAGING IV LINES

(71) Applicants: Timothy M. Gandee, Perry, OH (US); Heather M. Phyfer, Perry, OH (US)

(72) Inventors: Timothy M. Gandee, Perry, OH (US); Heather M. Phyfer, Perry, OH (US)

(21) Appl. No.: 16/242,588

(22) Filed: Jan. 8, 2019

(51) Int. Cl. A61M 5/14

(52) U.S. Cl. CPC A61M 5/1418; A61M 25/0606

(57) ABSTRACT

A spacer for separating multiple IV lines. The spacer includes a generally planar body having spaced apart holes formed through the body. Each hole has a diameter approximating an outer diameter of an IV line. The holes are operative to space apart at least two IV lines extending through at least two holes. Slits are formed through the body. The slits have a diameter that is smaller than the outer diameter of the IV line. Each slit extends from an outer edge of the body to a hole. The body is formed from a material that is sufficiently rigid to maintain an IV line in each hole and yet sufficiently resilient to move the IV line through the slit.

### BACKGROUND

The present exemplary embodiment relates to a medical spacer for separating intravenous (IV) lines when a patient is receiving multiple infusions. It finds particular application in a healthcare setting and will be described with particular reference thereto. However, it is to be appreciated that the present exemplary embodiment is also amenable to other like applications.

IV tubing is used to administer medications at a predetermined drip rate; to push multiple drip rate medications simultaneously; or to keep an IV line open or patent. IV tubing can come with no hubs or multiple hubs. Each hub can be used to connect another drip rate medication to a patient, or the opening can allow for one time doses or boluses to be manually pushed into the patient. Typically, a one-time bolus dose is administered in a hub of an IV drip that is a back-up flush—not a medication drip. Even when there are only two IV infusions—that is, one medication on a pump rate and one IV fluid flush line—medical error is possible if another medication is inadvertently pushed through the opposite or wrong line. For example, a bolus of the medication that was intended for the flush line can be wrongfully administered using the medication drip line. If the medication is, for example, a vasoactive drug or in a highly concentrated form, the error can compromise the patient's hemodynamics (or hemodynamic status), which can lead to instability of blood pressure or heart rate, etc. This instability is often momentary, but it can lead to a new medical crisis and side effects for the patient.

When multiple intravenous (IV) infusions are simultaneously administered to a patient, which is common with inpatient settings, the potential for tangled cords creates an even greater risk of medical error. A number of IV infusion errors have been observed in patients connected to multiple IV lines and fluid containers. Particularly, confusion or mistakes between IV lines lead to the misadministration of drugs. These errors are caused, for example, when a medical provider puts a one-time bolus medication into a hub on the wrong IV line or into a line that already has an incompatible medication moving through it. A reaction between the two incompatible medications can increase or decrease either drugs' potency and, sometimes, create a sediment that builds up inside the IV tube—either of which can affect the overall outcome that is intended for the patient being treated.

Instead of a pump, other medications may be administered using a roller clamp that is manually set by a medical provider. Even when the medical provider administers medications using the correct IV lines, medical error can result if he or she accidentally rolls a clamp open to the wrong drip. This might occur when the medical provider is working on other IV lines that are tangled or bunched up with the line that the roller clamp is on. This type of error is problematic for patients on high-alert medications who can be injured by being over- or under-medicated.

Nearly all errors reach the patient. For this reason, medical providers have to be diligent with distinguishing between the numerous IV lines; locating the primary infusion line; knowing what medications are going into each port; knowing where the flush line is; and knowing what drugs are being administered and at what rates; etc. Presently, there is no standard that is adopted by hospitals for managing the numerous IV lines that can potentially tangle, or for managing lines that are already tangled. To reduce the risks associated with multiple IV lines, hospitals raise awareness of IV medication errors among facility staff. Medical providers are recommended to trace existing lines from source to site and affix labels along the lines.

There is needed an article of manufacture or a medical supply that separates, organizes and/or prevents tangling or bunching up of IV lines. Particularly, a medical spacer is desired which can space apart multiple IV lines so that they are easily distinguishable from one another.

### BRIEF DESCRIPTION

One embodiment of the disclosure is directed to a spacer for organizing multiple IV lines. The spacer includes a generally planar body having spaced apart holes formed through the body. Each hole has a diameter approximating an outer diameter of an IV line. Slits are formed through the body. Each slit extends from an edge of the body to a hole. The body is formed from a material that is sufficiently rigid to maintain an IV line in each hole and yet sufficiently resilient to receive an IV line along the slit.

Another embodiment of the disclosure is directed to an article for separating multiple IV lines. The article includes a generally planar body and spaced apart holes formed through the body. Each hole has a diameter approximating an outer diameter of an IV line. The holes are operative to space apart at least two IV lines extending through at least two holes. The article further includes slits formed through the body. Each slit connects an outer edge of the body to a hole. Each slit has a width that is smaller than the outer diameter of the IV line. The slit is operative to deliver an IV line to the hole.

### BRIEF DESCRIPTION OF THE DRAWINGS

FIG. 1 illustrates a sample use of a spacer of the present disclosure;

FIG. 2 is a front perspective view of the spacer shown in FIG. 1;

FIG. 3 is a top perspective view of a spacer according to one embodiment of the disclosure;

FIG. 4 is top elevation view of the spacer; and

FIG. 5 is a front elevation view of the spacer.

### DETAILED DESCRIPTION

The present disclosure is directed to a medical supply unit or article that prevents tangling and bunching up of patient IV lines when multiple lines are connected to a single patient. Particularly, the disclosure is directed to a medical spacer that may be used to separate IV lines that are connected to, or ready for connection to, a patient. The present disclosure is intended to reduce the risk of human error that is caused by infusion rate or IV line mix-ups.

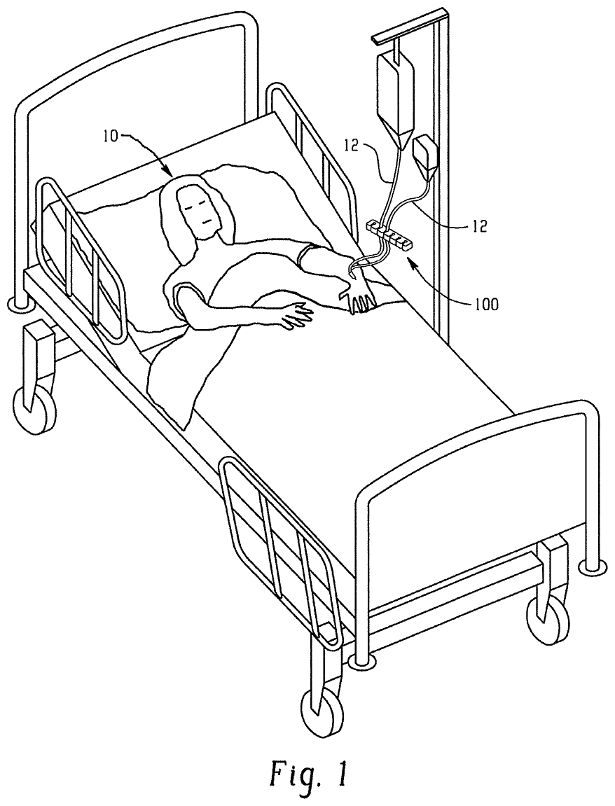

FIG. 1 illustrates an example scenario where the medical spacer of the present disclosure can be used. Intensive care patients, or patients undergoing surgical procedures, can have twelve or more infusion lines or fluid containers connected to them at once. In FIG. 1, a patient 10 is shown with multiple lines 12 attached to her. The IV lines 12 are spaced apart using the spacer 100 of the present disclosure. Although only one spacer 100 is shown in FIG. 1, multiple spacers can be used to maintain the spaced apart arrangement between the IV lines.

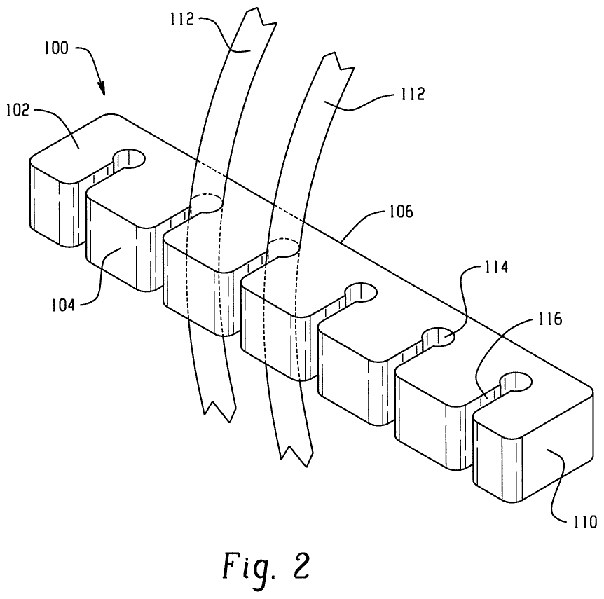

FIG. 2 is a blown-up front perspective view of the medical spacer 100 shown in FIG. 1. The medical spacer 100 is defined by a generally planar body 102. Although illustrated as a rectangular sheet, there is no limitation made herein regarding a general shape or profile of the spacer body 102. The exemplary spacer body 102 is defined by a first, front edge 104 and a second, back edge 106 that each extend along a longitudinal extent of the spacer body 102. Opposing first and second side edges 108, 110 extend along a lateral extent of the spacer body 102. In a preferred embodiment, the spacer body 102 is formed to have a uniform thickness, although medical spacers 100 of variable thickness are contemplated. The thickness can vary depending on the material used to manufacture the medical spacer 100. A medical spacer 100 should have sufficient thickness to prevent it from sliding about the IV lines 112 that it is connected to. Particularly, a thickness of the spacer body 102 is selected to provide enough friction contact between the outer circumference of the IV lines and holes in the spacer body 102 to maintain the medical spacer in position. A thickness between about 1/16^{th} inch to about 2 inches, and more preferably between about ¼-inch and 1 inch, and most preferably approximating ½-inch is contemplated for the embodiment.

Continuing with FIG. 2, the spacer body 102 includes at least two spaced apart holes 114 formed through the spacer body. Each hole 114 can accommodate one IV line 112. The illustrated spacer body 102 is shown with six holes 114, but there is no limitation made herein regarding the number of holes formed through one medical spacer. Each hole 114 has a diameter that approximates, or is negligibly larger than, the outer diameter of a conventional IV line 112 employed by the medical industry. Generally, two sizes of IV lines or tubes are employed in the medical industry: small IV lines having a 3-mm inner diameter and large IV lines having a 4-mm inner diameter. The disclosure contemplates holes 114 that are less than ¼-inch in diameter.

The spacer body 102 also include slits or channels 116 formed through the spacer body. Each slit 116 extends from the front edge of the spacer body 102 to a hole 114, although embodiments are contemplated where one slit can bifurcate into two or more branches that each terminate at a hole. Further embodiments are contemplated where at least one slit 116 extends from the front edge 104 of the spacer body 102 to a first hole 114 and at least one different slit extends from a different edge of the spacer body to a second hole. Each slit 116 extends from an edge 104-110 of the spacer body 102 to a hole 114 so that an IV line can be manually pushed through the slit to the hole or from the hole.

In operation, the holes 114 in the spacer body 102 separate and space apart at least two IV lines to prevent the lines from bunching or crossing over each other. For this reason, the spacer body 102 is formed from a material that is sufficiently rigid to maintain the IV lines in the holes 114, and yet sufficiently resilient to allow for the IV lines to move through the slits or channels 116. In one embodiment, the spacer body 102 can be formed from a light weight sponge rubber foam or like material. For example, the spacer body 102 can be formed from an EPDM/NeopreneSBR foam sheet. In the preferred embodiment, the spacer body 102 is formed from a light duty blended EPDM foam sheet.

In operation, the medical spacer 100 of the present disclosure can be manufactured from an inexpensive, disposable material. In the contemplated embodiment, the spacer body 102 is a consumable article that is formed from a material that can hold ink or sticker labels. In one embodiment, the medical spacer 100 can receive markings from a medical professional for identifying the IV line that is connected to a hole 114 or the medication associated with the IV line. In this manner, medical spacers 100 can be discarded after use and new medical spacers can be used for each patient or for new infusions.

Continuing with FIG. 4, the exemplary medical spacer 100 is shown to have tabs 118 that are defined by adjacent sides or walls of neighboring slits 116. The tabs 118 are a byproduct of the design of the spacer body 102. However, the spacer body 102 is desirably formed of a material that would allow markings to be placed on or adhered to the tabs 118, such as labels identifying the line moving through the hole 114 next to the tab. In one embodiment, the distance between adjacent holes 114 and/or slits 116 can approximate ¾-inch, although there is no limitation made herein on the size of the tab. Generally, the size of the tabs 118 are based on the number of holes 114 in the spacer body 102 and the distance between adjacent holes.

Another area that is available on the spacer body 102 for marking is the continuous top surface (continuous region 120) of the spacer body that is located between the holes 114 and the back edge 106. In the contemplated embodiment, the continuous region 120 has a width that approximates ½ to ¼ the total width of the spacer body 102. In one example embodiment, the width (or lateral edge 108, 110) of the spacer body 102 can approximate one inch, and the area located between the holes 114 and the back edge 106 can be about ¼-inch to about ½-inch and, preferably ¼-inch. The continuous region 120 of the spacer body 102 is a width that provides sufficient strength and rigidity to the spacer body, so that the spacer body does not tear when IV lines, which are being maintained by the medical spacer 102, are being handled or moved by a medical practitioner.

Returning to FIG. 1, the medical spacer 100 of the present disclosure is operative to self-suspend from at least two IV lines 12 extending through at least two holes in the spacer body. FIG. 2 shows in greater detail several IV lines 112 being maintained by the medical spacer 100 in FIG. 1. As illustrated, a hole 114 is operative to surround an outer circumference or profile of the IV line and to selectively and removably hold the IV line using an interference fit. As disclosed supra, the holes 114 are of a diameter substantially matching the outer diameter of the IV line intended for being maintained by the medical spacer 100. In one embodiment, the holes 114 are of uniform diameter for accommodating large IV lines. In another embodiment, the holes 114 are of uniform diameter for accommodating small IV lines. Although not illustrated, embodiments are also contemplated where the spacer body 102 includes holes 114 of two different sizes: at least one hole to accommodate a large IV line and at least a second hole to accommodate a small IV line.

Returning to FIG. 4, the diameter of each hole 114 is greater than a width of each slit 116. As also disclosed supra, the spacer body 102 is formed from a material that is sufficiently resilient to allow for an IV tube 112 to be manually pushed through the slit 116, but to return to form when the IV tube is not in the slit. Therefore, the walls 122 defining a channel 116 (FIGS. 2, 5) deform when the outer circumference of an IV line is pressing against them. The width of the slit 116 must be wide enough to allow for an IV line to move or squeeze through the slit without deforming the IV tube, such as by collapsing the IV tube wall inward by pressing against it. Yet, the width of the slit 116 must be small enough to prevent IV tubes from shifting away from the hole 114, unless the IV tubes are manually moved from the hole.

In one embodiment, the width of a slit is uniform along the entire longitudinal extent of the slit or channel. In one embodiment, the width of the slit can vary. In such an embodiment, the slit can be wider at the front edge of the spacer body than where it meets the hole. In one embodiment, the shortest width along the slit can be where the slit meets the hole. In a preferred embodiment, the width of the each slit 116 is small enough to maintain the IV line 112 in a hole 114. While the interference fit generally maintains the IV line 112 in a hole 114, essentially the walls 122 defining the slit 116 can prevent the IV line from moving out of the hole. The width of the slit 116 can vary depending on the size of the IV tube being placed in the medical spacer 100. In one embodiment, a width along the entire slit 116 can approximate 0.125 inches. In one embodiment, a width of the slit 116 at its widest point can approximate 0.125 inches. In one embodiment, a width of the slit 116 at its least wide point can approximate 0.125 inches.

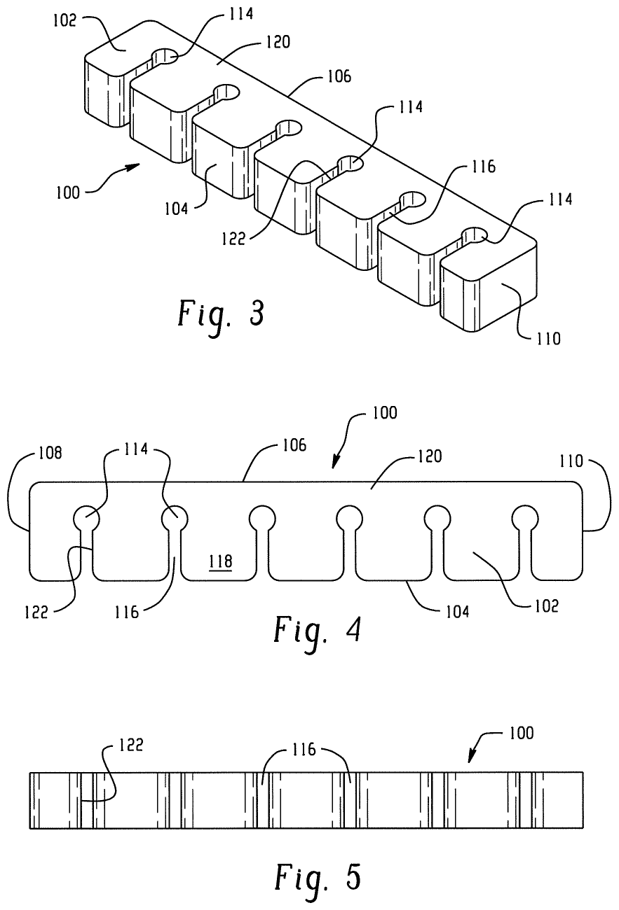

FIGS. 2-5 further illustrate one embodiment of a medical spacer 102 having holes 114 that are equally spaced apart along the longitudinal extent of the spacer body 102. The distance between two holes 114 can be based on the size of the IV lines 112 that are being separated by the medical spacer 100 and the number of IV lines that the medical spacer is intended to maintain. The illustrated embodiment shows equal sized holes 114 that are in linear alignment on the spacer body 102. However, embodiments are contemplated where alternating holes 114 can be offset. In one embodiment where the medical spacer 100 is intended to accommodate both large and small IV lines, the larger holes can be offset from the smaller holes relative to the front edge.

The holes 114 are in alignment along a longitudinal extent of the spacer body 102, and the slits 116 are formed transverse the longitudinal extent—that is, along the lateral extent. However, there is no limitation made herein regarding the location of the holes 114 and corresponding slits 116. The medical spacer 100 is intended to reduce the potential tangling of IV lines; to prevent IV lines from bunching up or crossing over; and to make it easier for the medical practitioner to retrace existing lines from source to site.

A method for using the medical spacer of the contemplated embodiment is now described. When attaching IV lines to patients, a medical practitioner can connect an IV tube to the medical spacer by pushing the tube through a slit to the hole, and then pushing a different IV tube to another hole in the same spacer. Multiple spacers can be placed between the fluid bags and the patient to continue the spaced arrangement between IV tubes. The medical professional can optionally mark the tab next to the hole with information regarding the medication associated with the line. When a line needs to be removed, the medical professional can manually pull the line from the hole using the channel, or push the line away from the hole through the channel. When the patient requires no more IV lines or medications, the medical spacer can be discarded or reused.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A medical spacer for organizing multiple intravenous (IV) lines, the medical spacer comprising:
a generally planar body;
spaced apart holes formed through the body, each hole having a diameter approximating an outer diameter of an IV line;
slits formed through the body, each slit extending from an edge of the body to a hole, each slit being operative to receive an associated IV line;
wherein the holes are operative to space apart at least two associated IV lines.

2. The medical spacer of claim 1, wherein the body is formed from a material that is sufficiently rigid to maintain said IV line in a hole and sufficiently resilient to receive said IV line in a slit.

3. The medical spacer of claim 1, wherein the body is formed from a sponge rubber foam material.

4. The medical spacer of claim 1, wherein the body is formed from a disposable material.

5. The medical spacer of claim 1, wherein the body is formed of a material that can hold ink or sticker labels.

6. The medical spacer of claim 1, wherein the medical spacer is operative to self-suspend from said at least two IV lines extending through at least two holes.

7. The medical spacer of claim 1, wherein a hole is operative to surround a circumference of said IV line and hold said IV line using an interference fit.

8. The medical spacer of claim 1, wherein the holes are equally spaced apart along a longitudinal extent of said body.

9. The medical spacer of claim 1, wherein a distance between two holes is based on a size of said at least two IV lines being separated by the medical spacer.

10. The medical spacer of claim 1, wherein the holes are of uniform diameter, the each hole having the diameter for receiving an associated large IV line.

11. The medical spacer of claim 1, wherein the holes are of uniform diameter, the each hole having the diameter for receiving an associated small IV line.

12. The medical spacer of claim 1, wherein the holes are of two different size diameters, the each hole having one of a first diameter for receiving an associated small IV line and a second diameter for receiving an associated large IV line.

13. The medical spacer of claim 1, wherein the diameter of the each hole is greater than a width of the each slit.

14. The medical spacer of claim 1, wherein a width of the each slit is small enough to maintain the associated IV line in a hole.

15. The medical spacer of claim 1, wherein the holes are in linear alignment on the body.

16. The medical spacer of claim 1, wherein the holes are spaced along a longitudinal extent of the body and the slits are formed along a lateral extent of the body.

17. The medical spacer of claim 1, wherein the each slit has a uniform width from the edge of the body to the hole.

18. An article for separating multiple intravenous (IV) lines, the article comprising:
a generally planar body;
spaced apart holes formed through the body, each hole having a diameter approximating an outer diameter of an IV line, wherein the holes are operative to space apart at least two associated IV lines extending through the holes;
slits formed through the body, each slit connecting an edge of the body to a hole, the each slit having a diameter that is smaller than the outer diameter of the IV line;
wherein the slit is operative to deliver an associated IV line to a hole.

19. The article of claim 18, wherein the body is formed from a material that is sufficiently rigid to maintain said IV line in the hole and sufficiently resilient to receive said IV line through the slit.
